(19) United States
(12) Patent Application Publication
(10) Pub. No.: US 2026/0069499 A1
(43) Pub. Date: Mar. 12, 2026

(54) LIGHT-BASED FEEDBACK OUTPUT BY ACCESSORY DEVICES

(71) Applicant: Stryker Corporation, Portage, MI (US)

(72) Inventors: Taylor Christian Sears, Bellingham, WA (US); Tyson G. Taylor, Bothell, WA (US)

(73) Assignee: Stryker Corporation, Portage, MI (US)

(21) Appl. No.: 19/320,934

(22) Filed: Sep. 5, 2025

Related U.S. Application Data
(60) Provisional application No. 63/691,813, filed on Sep. 6, 2024.

(51) Int. Cl. A61H 31/00; A61N 1/39

(52) U.S. Cl. CPC A61H 31/005; A61N 1/3904; A61N 1/3993; A61H 2201/0188; A61H 2230/655

(57) ABSTRACT

Example techniques relate to an accessory device outputting light-based feedback related to a treatment or physiological condition of a subject. An example method includes identifying a treatment parameter indicative of a treatment being administered to a subject; determining that the treatment parameter is outside of a threshold range; and in response to determining that the treatment parameter is outside of the threshold range, causing an accessory device to output a light signal indicative of a recommendation to alter the treatment.

### CROSS-REFERENCE TO RELATED APPLICATION

This application claims priority to U.S. Provisional Application No. 63/691,813, which was filed on Sep. 6, 2024 and is incorporated by reference herein in its entirety.

### BACKGROUND

In the event of a patient experiencing a sudden medical emergency, rescuers are deployed to the location of the person to provide assistance and potential transport. Rescuers utilize portable medical devices, such as monitor-defibrillators and mechanical chest compression devices, to monitor and treat the patient. If the patient needs additional medical care, the rescuers transport the person to a clinical environment. The portable medical devices can utilize removably connected accessory devices to monitor and treat the patient. For instance, a blood pressure cuff can plug into a monitor-defibrillator during use and can report blood pressure measurements of the patient to the monitor-defibrillator.

### BRIEF DESCRIPTION OF THE DRAWINGS

FIG. 1 illustrates an example environment utilizing on-accessory feedback to facilitate treatment and monitoring of a subject.

FIG. 2 illustrates an example environment of a medical device configured to output light-based feedback on an accessory device.

FIGS. 3A and 3B illustrate example accessory devices configured to output feedback. FIG. 3A is a cross-sectional view of an example electrode pad that can report feedback. FIG. 3B is a view of an example oximetry sensor that can report feedback.

FIG. 4 illustrates an example process for communicating feedback regarding a treatment via an accessory device.

FIG. 5 illustrates an example process for communicating physiological feedback via an accessory device.

FIG. 6 illustrates an example of an external defibrillator configured to perform various functions described herein.

FIG. 7 illustrates a chest compression device configured to perform various functions described herein.

### DETAILED DESCRIPTION

Various implementations described herein relate to providing on-accessory feedback using visual signals, such as light signals. In some cases, a medical device (e.g., a monitor-defibrillator) is configured to identify that a patient has a physiological condition (e.g., an arrhythmia) that can be addressed by a treatment (e.g., electrical shock). In some examples, the medical device monitors an ongoing treatment (e.g., chest compressions) administered to the patient. Based on an analysis of one or more parameters, such as physiological parameters, the medical device generates feedback based on the physiological condition or the treatment. For example, the feedback may indicate that the condition of the patient is deteriorating, the treatment is being administered too slowly, or the like. The medical device itself, may output the feedback in the form of visual alerts, such as on a display screen of the medical device. A rescuer, for instance, alters a treatment, or administers a new treatment, based on the feedback.

However, if the patient is experiencing a medical emergency, the rescuer may be focused primarily on the patient. The rescuer may be looking at the patient, rather than the medical device, in order to catch any sudden changes in the patient's condition. For example, the rescuer is looking at the patient's chest while administering manual chest compressions. As a result, the rescuer is unable to see the visual alerts on the display screen of the medical device, and is therefore unable to effectively respond to the feedback.

In various implementations of the present disclosure, visual feedback is output by an accessory device connected to the patient. For example, electrodes adhered to the patient's chest output a light signal indicating the feedback to the rescuer. Because the rescuer is focused on the body of the patient during the medical emergency, the rescuer is able to ascertain the feedback more efficiently if it is output by the accessory device rather than medical device connected to the accessory device.

Implementations of the present disclosure are directed to improvements in the field of medical devices. By outputting feedback from devices attached to the patient, the patient's condition can be ascertained by the rescuer, and can be addressed by the rescuer, more efficiently than feedback output from devices that are located at a distance from the patient.

Implementations of the present disclosure will now be described with reference to the accompanying figures.

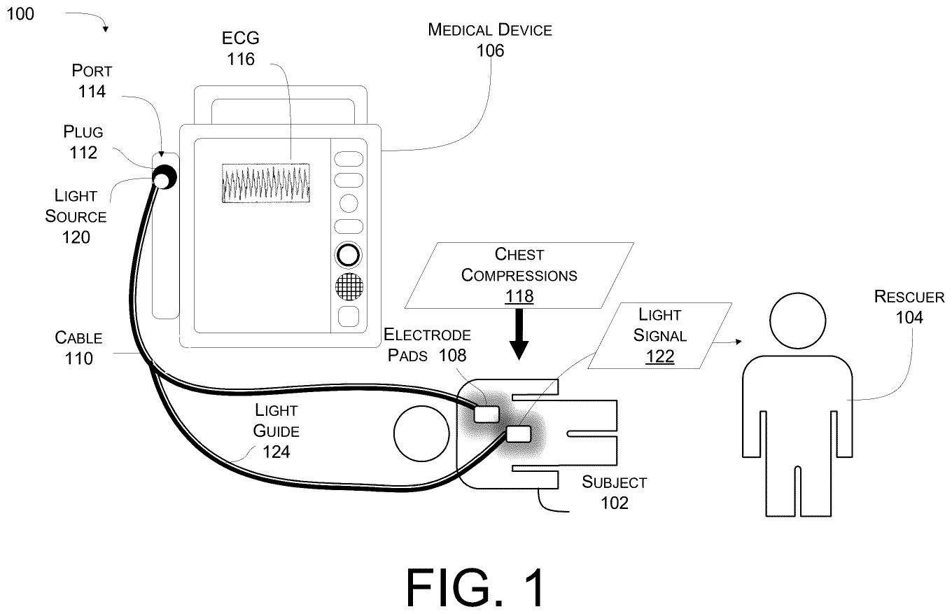

FIG. 1 illustrates an example environment 100 utilizing on-accessory feedback to facilitate treatment and monitoring of a subject 102. In various cases, the subject 102 is experiencing a medical emergency within the environment 100. For example, the subject 102 may be experiencing cardiac arrest or some other type of acute medical condition.

The environment 100, for example, is a non-clinical environment. In some cases, the environment 100 is outside of a hospital, medical clinic, or other facility with ample equipment and infrastructure to monitor and treat the subject 102. In various cases, the condition of the subject 102 is severe and time-sensitive, such that it is beneficial that the subject 102 receives monitoring and/or treatment before being transported to a clinical environment.

In various cases, a rescuer 104 is present in the environment 100 to perform monitoring and treatment of the subject 102. In some cases, the rescuer 104 is an emergency medical service (EMS) provider with specific training for medical emergencies. In some examples, the rescuer 104 is a bystander within the environment 100, and optionally lacks specific medical training.

The rescuer 104, in various implementations, operates a medical device 106 that facilitates monitoring and/or treatment of the subject 102. The medical device 106, for instance, is a portable device that is configured to operate outside of a clinical environment. In various cases, the medical device 106 includes an on-board power source that can be used in environments without mains power availability. For example, the medical device 106 includes one or more batteries configured to provide power to components within the medical device 106 during operation in the environment 100.

The medical device 106 is configured to administer a treatment and/or identify a condition of the subject 102. In particular cases, the medical device 106 is a portable patient monitor. For instance, the medical device 106 is a monitor-defibrillator and/or automated external defibrillator (AED). In some examples, the medical device 106 is another type of portable medical device, such as a mechanical chest compression device, a portable ventilator, a portable ultrasound monitor, a flow monitor, or a portable patient monitor of any type.

The medical device 106 is configured to be coupled to one or more accessory devices. As used herein, the term “accessory device” may refer to any apparatus that can be used in conjunction with a primary device, thereby enabling functions of the primary device. In various cases, the accessory device(s) are separate from, but can be connected to, the medical device 106. The accessory device(s), in some cases, are removably coupled with the medical device 106 via one or more plugs and ports. In some examples, the accessory device(s) are wirelessly coupled to the medical device 106. In some cases, the accessory device(s) include one or more disposable devices, such that one instance of an accessory device may be used with the subject 102 and another instance of the same type of accessory device may be used with a different subject. In various cases, the accessory device(s) are communicatively coupled with the medical device 106.

In various cases, the medical device 106 detects one or more physiological parameters of the subject 102 via the accessory device(s). The terms “physiological parameter,” “parameter,” and their equivalents, may refer to a metric that is indicative of a condition of a subject's body. Examples of physiological parameters include, for instance, an electrocardiogram (ECG), a blood pressure, a blood oxygenation (e.g., regional oxygenation, pulse oxygenation, plethysmograph, etc.), an airway parameter (e.g., a capnograph, a partial pressure of CO2, a partial pressure of O₂, a tidal volume, flow rate through an airway, airway pressure, respiration rate, etc.), a temperature (e.g., a core temperature, a temperature of an extremity, etc.), a blood flow (e.g., a blood velocity, blood flow rate, etc.), a heart rate, a pulse rate, a motion (e.g., an acceleration), a concentration of a chemical in a body fluid (e.g., blood glucose), or any combination thereof. In some examples, the accessory device(s) include one or more sensors and are configured to report data indicative of one or more parameters to the medical device 106. For instance, the accessory device(s) include an electrode, a blood pressure cuff, an ultrasound-based blood pressure monitor, a pulse oximeter, an oximetry sensor, an airway sensor (e.g., a capnography sensor, a carbon dioxide sensor, an oxygen sensor, etc.), a thermometer, an ultrasound-based blood flow sensor, a pulse sensor, an accelerometer, a gyroscope, a chemical sensor, or any combination thereof.

In some cases, the accessory device(s) are configured to output a treatment to the subject 102 in response to one or more signals from the medical device 106. For example, the accessory device(s) include electrodes configured to output an electrical signal to the subject 102 (e.g., electric shocks, pacing pulses, etc.). In some cases, the accessory device(s) include a plunger or band structure configured to administer chest compressions. In some examples, the accessory device(s) include a laryngoscope, airway adaptor, or ventilation device configured to administer positive pressure ventilation to the subject 102.

The accessory device(s) are powered by the power source(s) of the medical device 106, in some examples. In some cases, the accessory device(s) are powered based on at least one on-board power source. For example, one or more batteries, capacitors, or other power storage devices may be integrated into the medical device 106 and/or the accessory device(s) to power circuitry within the accessory device(s)

In particular implementations of FIG. 1, electrode pads 108 are examples of the accessory device(s) of the medical device 106. In various cases, multiple electrode pads 108 are adhered to the skin of the subject 102. Each of the electrode pads 108, for instance, includes an electrically insulative substrate, an electrically conductive hydrogel disposed on the electrically insulative substrate, and an electrode layer disposed between the electrically insulative substrate and the electrically conductive hydrogel. The electrode layer, for instance, includes a metal (e.g., silver). In various implementations, the electrode pads 108 are disposed on the chest of the subject 102.

In various examples, the electrode pads 108 receive power and transmits data to the medical device 106 via a wired interface. The electrode pads 108 are electrically coupled with the medical device 106 via a cable 110. The medical device 106 may detect a relative electrical potential between the electrode layers of the electrode pads 108 over time, due to an electrical signal output by the heart of the subject 102. Accordingly, the electrode pads 108 configured to detect an ECG of the subject 102. The cable 110 is connected with a plug 112 that is configured to be removably connected with a port 114 of the medical device 106.

Specifically, the port 114 is configured to receive the plug 112 of the electrode pads 108. As used herein, the term “plug,” and its equivalents, may refer to a physical connector that can be removably inserted into a port, and which electrically connects the port to a circuit coupled to the plug when the plug is physically inserted into the port. The plug 112 of the electrode pads 108 is configured to be removably coupled with the port 114 of the medical device 106. For example, the plug 112 may be configured to be magnetically coupled, snap-fit, or tension-fit into the shape of the port 114. In various cases, the plug 112 includes one or more conductive contacts that are configured to be disposed against one or more conductive contacts of the port 114 when the plug 112 is connected to the port 114. Accordingly, electrical signals can be transmitted between the plug 112 and the port 114 when the plug 112 is connected to the port 114.

The plug 112 is connected to the cable 110, which is configured to carry electrical signals. In various cases, the cable 110 includes one or more elongated conductive wires that are electrically coupled to the plug 112. The conductive wire(s) are configured to carry the electrical signals to and/or from the plug 112. In some cases, the cable 110 further includes an insulative covering. In some cases, the covering includes a polymer, a rubberized material, a woven material, or the like. The cable 110 is flexible, in various implementations.

The medical device 106, for instance, is configured to detect an ECG 116 of the subject 102 using the electrode pads 108. The electrode pads 108 detect an electrical signal output by the heart of the subject 102. In various cases, the electrode pads 108 transmit a signal to the medical device 106 via the cable 110 that is indicative of a magnitude of the electrical signal over time. The signal, for instance, may be an analog signal or a digital signal. The medical device 106, for instance, derives the ECG 116 based on the electrical signal received from the electrode pads 108. The ECG 116 includes data indicative of an electrical potential output by the heart of the subject 102 over time.

The ECG 116 is indicative of a condition of the subject 102. For example, in some cases, the ECG 116 indicates whether the subject 102 has an arrhythmia. Shockable arrhythmias, such as ventricular fibrillation (VF) or ventricular tachycardia (VT) are treatable by administering an electrical shock to the heart of the subject 102. Bradycardia, for instance, is a type of arrhythmia that is treatable by pacing. In various cases, the medical device 106 includes a display that visually presents the ECG 116 to the rescuer 104. Accordingly, the medical device 106 allows the rescuer 104 can ascertain whether the subject 102 is in need of a treatment, such as an electrical shock or pacing, by viewing the medical device 106.

The medical device 106 is configured to output other types of feedback to the rescuer 104, in some cases. As used herein, the term “feedback,” and its equivalents, refers to any information that is relevant to monitoring and/or treatment of a subject. Physiological parameters, such as the ECG 116, represent one type of feedback that the medical device 106 is configured to provide the rescuer 104. In some cases, the medical device 106 also provides feedback related to a treatment that has been administered to the subject 102.

For instance, in some examples, the subject 102 is receiving chest compressions 118. The chest compressions 118, in some cases, are administered manually by the rescuer 104. In some examples, the chest compressions 118 are administered by a mechanical chest compression device. In some cases, the subject 102 has an arrhythmia (such as VF) that prevents the heart of the subject 102 from effectively pumping blood through the body of the subject 102. If administered appropriately, the chest compressions 118 induce blood flow in the body of the subject 102, even if the heart of the subject 102 is functioning insufficiently to spontaneously circulate blood. Accordingly, the chest compressions 118 are capable of preventing or reducing hypoxic injury to the subject 102 when the subject lacks spontaneous circulation. Other treatments that could improve a condition of the subject 102 include, for instance, administration of an electrical shock (e.g., a defibrillating shock), pacing pulses, assisted ventilation, one or more medications, and the like.

The quality of treatments, such as the chest compressions 118, can be dependent on characteristics of the treatments as-administered. These characteristics are referred to herein as “treatment parameters.” Examples of treatment parameters include a magnitude, frequency, position, or duty cycle of a treatment. For instance, the quality of the chest compressions 118 is dependent on their depth, frequency, pressure, position (e.g., as-applied on the chest of the subject 102), and duty cycle. In some examples, the quality of the chest compressions 118 is dependent on whether the chest compressions 118 achieve adequate recoil. For example, the chest of the subject 102 should be fully released between the chest compressions 118 in order to achieve high-quality chest compressions. Examples of treatment parameters that are relevant to recoil include chest compression depth over time, pressure over time, and the presence or absence of an applied pressure on the chest between compressions.

In various cases, the medical device 106 is configured to detect one or more treatment parameters and to display the treatment parameter(s). By recognizing the treatment parameter(s), in some cases, the rescuer 104 can adjust the administration of the treatments to enhance the condition of the subject 102. In some implementations, the medical device 106 receives a communication signal indicative of the treatment parameter(s). In some cases, the medical device 106 derives the treatment parameter(s) based on data indicative of a parameter detected by one or more sensors. For instance, the medical device 106 may determine the frequency of the chest compressions by receiving data from a sensor device (not illustrated) configured to detect an acceleration or pressure while disposed on the chest of the subject 102. In some cases, the sensor device is integrated with the electrode pads 108. According to some examples, the medical device 106 is configured to infer the treatment parameter(s) based on the physiological parameter(s). For example, the medical device 106 is configured to infer the frequency of the chest compressions 118 by detecting a periodic chest compression artifact in the ECG 116.

In some implementations, feedback is in the form of one or more alarms. For instance, in some cases, the medical device 106 is configured to analyze the physiological parameter(s) (e.g., the ECG 116) and/or the treatment parameter(s) (e.g., the frequency of the chest compressions 118). In various implementations, the medical device 106 compares the physiological parameter(s), the treatment parameter(s), a rate-of-change of the physiological parameter(s) with respect to time, a rate-of-change of the treatment parameter(s) with respect to time, an amount of artifact of the physiological parameter(s), a metric derived based on one or more parameters (e.g., a metric representing the likelihood that the ECG 116 is indicative of VF or VT), or any combination thereof, to one or more thresholds. If any of these metrics is lower than a first threshold, or is higher than a second threshold, then the medical device 106 is configured to generate an alarm signal. The alarm signal, for instance, may notify the rescuer 104 of an acutely important aspect of the condition of the subject 102 or the treatment administered to the subject 102. In some cases, the alarm signal indicates that a heart rate of the subject 102 is below a threshold, that a depth of the chest compressions 118 is below a threshold, or the like. In various cases, the medical device 106 displays the alarm signal, so that the rescuer 104 can take immediate corrective action upon viewing the alarm signal. In some examples, the alarm signal is indicative of errors or artifact in the physiological parameter(s), in addition to acute medical conditions of the subject 102. For example, if a signal-to-noise ratio (SNR) of the physiological parameter(s) is lower than a threshold, the medical device 106 is configured to generate an alarm signal.

According to some examples, the medical device 106 provides feedback that coaches the rescuer 104 through administering a treatment to the subject 102. For example, in cases in which the medical device 106 determines that the chest compressions 118 should be administered to the subject, the medical device 106 may communicate a timing of the chest compressions 118 that enables the rescuer 104 to manually administer the chest compressions 118 in an appropriate manner.

In some cases, the medical device 106 provides feedback that is relevant to the function of the medical device 106 or the accessory device(s) themselves. According to some instances, the medical device 106 reports when an accessory device is functioning improperly. For example, if the medical device 106 detects that the ECG 116 is unreliable (e.g., by detecting that a significant artifact in the ECG 116 is indicative of improper placement or attachment of the electrode pads 108 on the chest of the subject 102), then the medical device 106 outputs, to the rescuer 104, feedback indicating an instruction to review or reattach the electrode pads 108. In some cases the reliability of a physiological parameter is identified based on a signal-to-noise ratio of data indicative of the physiological parameter. Other feedback indicative of the state of the medical device 106 or the accessory device(s) includes indicators of low battery levels, electrical disconnections, communication interruptions (e.g., interruption of a wireless communication channel associated with the medical device 106 or accessory device(s)), calibration, calibration errors, sensor failures (e.g., an indication that an obstruction exists in a filter line of a capnograph sensor that prevents accurate measurements), errors with unknown causes, or the like.

In general, the medical device 106 displays various forms of feedback, which can be viewed by the rescuer 104. However, in high-stress rescue scenarios, it may be difficult for the rescuer 104 to constantly view the medical device 106. For instance, if the rescuer 104 is administering the chest compressions 118 to the subject 102, then the rescuer 104 may be primarily watching the chest of the subject 102 to ensure that the chest compressions 118 are administered appropriately. In some cases, it may be beneficial for the rescuer 104 to watch the body of the subject 102 for signs of consciousness, breathing, or other changes in the condition of the subject 102. The visual attention of the rescuer 104 on the body of the subject 102, for instance, may prevent the rescuer 104 from viewing important feedback displayed by the medical device 106.

In some cases, the medical device 106 can address this problem by outputting non-visual alerts, such as audible alerts via a speaker. However, audible alerts have their own drawbacks. For instance, it may be difficult for the medical device 106 to simultaneously provide multiple types of feedback via audible alerts. Further, the rescuer 104, in some examples, may have to communicate with other people at the rescue scene to facilitate care of the subject 102, may monitor the subject 102 by listening to the subject 102 (e.g., by listening for agonal breathing), or the like. As a result, audible alarms may be insufficient to provide effective and complex feedback to the rescuer 104.

Various implementations of the present disclosure address these and other problems by visually outputting the feedback on the accessory device(s), rather than the medical device 106. In various implementations, the medical device 106 activates a light source 120 that outputs a light signal 122 indicative of one or more types of feedback. The light source 120, for instance, includes a LASER, an incandescent bulb, a fluorescent light source, a light-emitting diode (LED), a chemiluminescence source, an electrochemiluminescence source, or any combination thereof. In some cases, a chemical reaction occurs within the light source 120 that causes the production of the light signal 122. For example, the chemical reaction may include a reaction between phenyl oxylate ester, hydrogen peroxide, and a fluorescent dye. As a result of the reaction, the fluorescent dye is energized and phenol and carbon dioxide are produced as a byproduct. In particular cases, the light source 120 includes one or more LEDs. In various implementations of the present disclosure, the light signal 122 (or any other light signal described herein) can include, or be substituted with, any visual signal or visual indicator, such as a liquid crystal display (LCD) signal or an e-ink signal.

In various cases, the light source 120 is integrated with the port 114 of the medical device 106 and/or the plug 112 of the accessory device. Accordingly, the medical device 106, in some cases, is configured to activate the light source 120 by outputting an electrical signal (e.g., a voltage or electrical current) to the light source 120.

In implementations in which the light source 120 is located within, or proximate to, the medical device 106, the light signal 122 may be transported to the electrode pads 108 via a light guide 124. In various cases, the light guide 124 is a fiber-optic cable that is disposed on, or integrated with, the cable 110. In some cases, the light guide 124 includes a core that transmits the light signal 122 from the light source 120 to the electrode pads 108. The core, for instance, includes a light-transmissive material, such as a transparent polymer (e.g., polymethyl methacrylate (PMMA)). In some cases, the light guide 124 includes a cladding layer disposed on the core. Optionally, the light guide 124 includes a cover, such as a woven fabric. In some examples, the light guide 124 is designed to maximize total internal reflection, such that a significant amount of the light signal output by the light source 120 is output by an end of the light guide 124 disposed at the electrode pads 108. In some examples, the light guide 124 is designed to leak the light signal 122 along the length of the light guide 124, such that the light guide 124 itself outputs a portion of the light signal 122 along the length of the cable 110. In some cases, the light guide 124 terminates at a transparent bulb (e.g., a solid shape that transmits and outputs light) disposed on the electrode pads 108. The bulb, for instance, includes polycarbonate. For instance, the bulb outputs the light signal 122 multidirectionally from the electrode pads 108, such that the light signal 122 is viewable by the rescuer 104 from multiple directions.

Although FIG. 1 illustrates that the light source 120 is located at the plug 112 and/or the port 114, implementations are not so limited. In some examples, the light source 120 is integrated with the electrode pads 108. For example, the medical device 106 is configured to activate the light source 120 by outputting an electrical signal indicative of the feedback to the electrode pads 108 along the cable 110.

The light signal 122 indicates the feedback in various ways. In some cases, a color of the light signal 122 is indicative of the feedback. For example, the rescuer 104 may recognize that a physiological parameter of the subject 102, or a parameter of the chest compressions 118, is within a predetermined range when the light signal 122 is green; whereas the rescuer 104 may recognize that the physiological parameter or the parameter of the chest compressions 118 is outside of the predetermined range when the light signal 122 is red. In some examples, the light signal 122 is pulsed (e.g., the light source 120 “blinks”). A pulse pattern of the light source 120, a frequency of the pulses, a duty cycle of the pulses, or any combination thereof, is indicative of the feedback in some cases. For instance, the light signal 122 is pulsed at a frequency associated with a recommended timing of the chest compressions 118. In some cases, an intensity of the light signal 122 is indicative of the feedback. For example, as the physiological parameter of the subject 102 trends farther outside of the predetermined range, the light signal 122 may increase in intensity. In some cases, multiple light sources 120 are included (e.g., along the cable 110), wherein the subset of light sources 120 that are illuminated are indicative of a particular type of feedback.

Various implementations of the present disclosure enable different types of rescuers (e.g., including the rescuer 104) in the environment 100 to focus on different types of feedback. For example, if the subject 102 has a condition (e.g., cardiac arrest), the multiple rescuers manage different aspects of care to the subject 102. For instance, the rescuer 104 may be responsible for administering the chest compressions 118 to the subject 102, and another rescuer may be responsible for administering positive pressure ventilation to the subject 102 (e.g., via a bag-valve mask). By outputting feedback related to the chest compressions 118 via the light signal 122 to the rescuer 104, the other rescuer may be prevented from being distracted by the chest compression feedback, which is irrelevant to the other rescuer's specific responsibilities in the rescue scene. In some cases, the bag-valve mask provides light-based feedback to the other rescuer about the positive pressure ventilation. Accordingly, some implementations of the present disclosure prevent different rescuers in a rescue scene from being distracted by feedback intended for other rescuers in the scene.

A specific example of the present disclosure will now be described with reference to FIG. 1. In various cases, the subject 102 has unexpectedly collapsed in an airport terminal. The rescuer 104 is deployed to the scene of the airport terminal with the medical device 106. After connecting the electrode pads 108 to the chest of the subject 102 and connecting the plug 112 to the port 114 of the medical device 106, the medical device 106 outputs feedback to the rescuer 104 instructing the rescuer 104 to manually administer the chest compressions 118 to the subject 102. In particular, the medical device 106 outputs the light signal 122 via the light source 120 and the light guide 124 indicating the recommended chest compressions 118. Additionally, the medical device 106 detects the ECG 116 of the subject 102 using the electrode pads 108.

At first, the rescuer 104 administers the chest compressions 118 too slowly to effectively pump blood through the body of the subject 102. The medical device 106 detects that the chest compressions 118 by analyzing the ECG 116 or transthoracic impedance. The medical device 106 determines that the frequency of the chest compressions 118 is outside of a predetermined range associated with effective CPR. In response, the medical device 106 outputs the light signal 122 with a red color. Moreover, the medical device 106 coaches the rescuer 104 into administering the chest compressions 118 at an appropriate rate by causing the light signal 122 to blink at a frequency associated with the predetermined range. Because the rescuer 104 is watching their hands as they are administering the chest compressions 118, they are able to notice the feedback and take corrective action. Upon bringing the chest compressions 118 into the appropriate range, the medical device 106 causes the light signal 122 to transition to a green color.

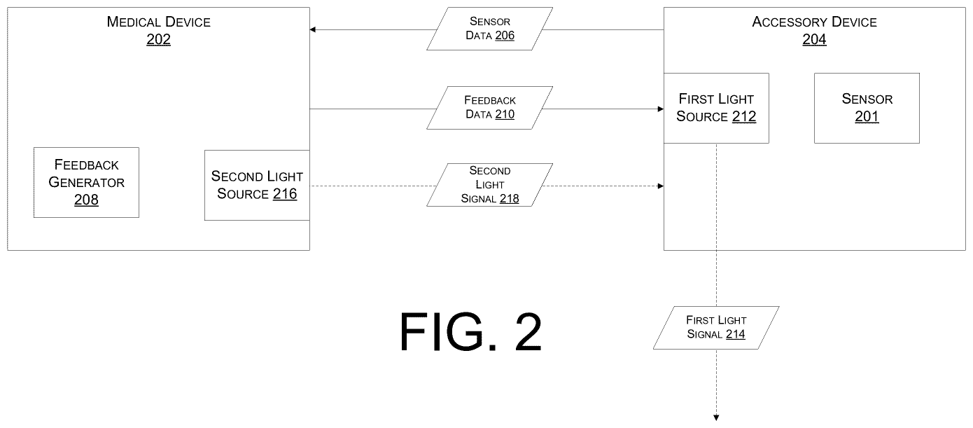

FIG. 2 illustrates an example environment 200 of a medical device 202 configured to output light-based feedback on an accessory device 204. In some cases, the medical device 202 is the medical device 106 and/or the accessory device 204 includes the electrode pads 108. In some cases, the medical device 202 is a patient monitor, a blood flow sensor, or a mechanical chest compression device.

According to some examples, the accessory device 204 includes a sensor 201 configured to detect a parameter. For instance, the sensor 201 may be an accelerometer or gyroscope configured to detect a movement of the accessory device 204. The parameter, in some cases, is a physiological parameter of a subject (e.g., a patient). For instance, the sensor 201 includes an electrode (e.g., an ECG electrode and/or an electroencephalogram (EEG) electrode), a heart rate sensor, a pulse rate sensor, a gas sensor (e.g., a CO₂ sensor, an O₂ sensor, or the like), a respiration sensor, a thermometer, a blood pressure sensor (e.g., a blood pressure cuff, an ultrasound-based blood pressure sensor, etc.), a blood oxygenation sensor (e.g., a pulse oximeter, a regional oxygenation sensor, a cerebral oxygenation sensor, etc.), or any combination thereof. The sensor 201, for instance, is configured to detect the parameter while being at least partially disposed outside of the body of the subject.

The accessory device 204 is configured to generate sensor data 206 based on the parameter detected by the sensor 201. The accessory device 204 is configured to transmit the sensor data 206 to the medical device 202. In some cases, the sensor data 206 is transmitted along a cable that extends between the medical device 202 and the accessory device 204. In some examples, the sensor data 206 is wirelessly transmitted to the medical device 202.

The medical device 202 includes or otherwise executes a feedback generator 208 that determines feedback associated with monitoring or treating a subject (e.g., a patient). Any type of feedback described herein can be generated by the feedback generator 208, such as feedback related to a condition of the subject, an efficacy of a treatment administered to the subject, coaching related to the treatment, or a status of the medical device 202 and/or the accessory device 204. In some examples, the feedback generator 208 utilizes the sensor data 206 to generate the feedback. In some cases, the feedback generator 208 generates the feedback based on one or more parameters, which may be detected by the accessory device 204 and/or one or more additional accessory devices.

In particular cases, the medical device 202 outputs feedback data 210 indicating the feedback. In some cases, the feedback data 210 is transmitted along the cable and/or a wireless interface.

The accessory device 204 includes a first light source 212 configured to output a first light signal 214 indicating the feedback. For instance, the accessory device 204 outputs the first light signal 214 based on the feedback data 210. For example, a color, pulse pattern, or brightness of the first light signal 214 may communicate the feedback.

In some implementations, the medical device 202 includes a second light source 216 configured to output a second light signal 218 indicating the feedback. The second light signal 218 is transmitted to the accessory device 204, such as via a light guide. For example, a color, pulse pattern, or brightness of the second light signal 218 may communicate the feedback.

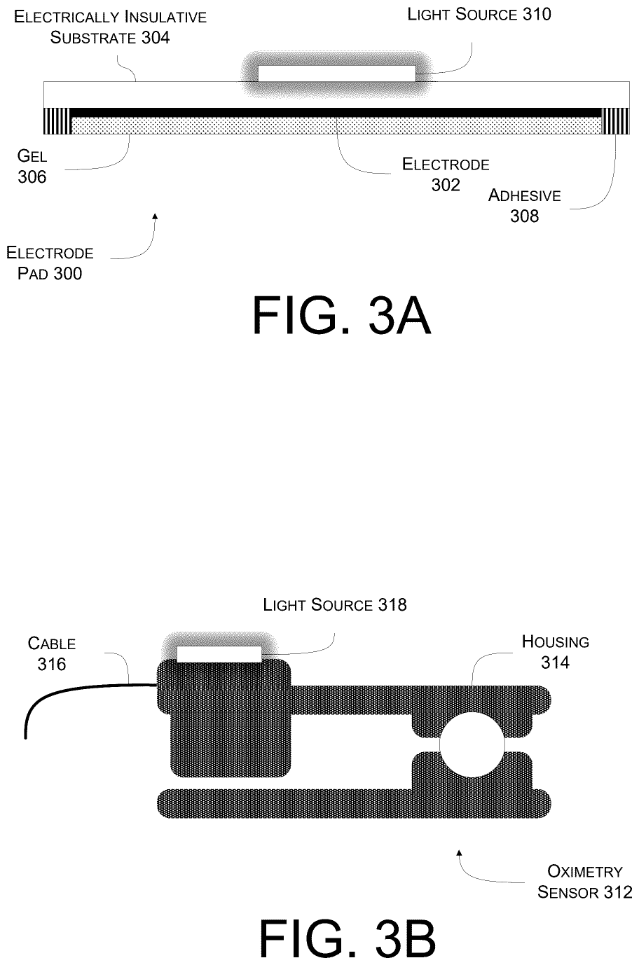

FIGS. 3A and 3B illustrate example accessory devices configured to output feedback. FIG. 3A is a cross-sectional view of an example electrode pad 300 that can report feedback. The electrode pad 300, for instance, is one of the electrode pads 108 described above.

The electrode pad 300 includes an electrode 302 that is configured to conduct electrical signals to and/or from a subject. The electrode 302, for instance, includes silver-silver chloride (Ag/AgCl), nickel, another conductive material, or any combination thereof. The electrode 302 is disposed on a surface of an electrically insulative substrate 304. For example, the electrically insulative substrate 304 includes a foam, a rubberized material, a polymer, or some other electrically insulative material. A gel 306 is disposed on the electrode 302, such that the electrode 302 is disposed between the electrically insulative substrate 304 and the gel 306. The gel 306 is electrically conductive. For instance, the gel 306 is a hydrogel including one or more electrolytes. An adhesive 308 is disposed on a perimeter of the surface of the electrically insulative substrate 304 that is disposed against the electrode 302, such that the adhesive 308 is disposed around a border of the electrode 302 and the gel 306. The adhesive 308 is configured to attach the electrode pad to the skin of a subject. For instance, the adhesive 308 is biocompatible.

In various implementations of the present disclosure, the electrode pad 300 further includes light source 310. The light source 310 is configured to output, to a user, light signal indicating feedback associated with monitoring and/or treating the subject. For instance, a frequency, durations, pulse patterns, periods, colors, tones, intensities, or any combination thereof, of the light signal is indicative of the feedback.

Although not specifically illustrated in FIG. 3A, the electrode 302 may be electrically coupled to one or more wires within a cable extending from the electrode pad 300. In some cases, the electrode 302 and the light source 310 are connected to a power source of the electrode pad, such as when the electrode pad 300 is disconnected from the medical device. In some examples, the electrode 302 and the light source 310 are electrically connected to a power source in the medical device when the electrode pad 300 is connected with the medical device. Thus, the electrode 302 and/or the light source 310 may be powered regardless of whether the electrode pad 300 is connected to the medical device.

FIG. 3B is a view of an example oximetry sensor 312 that can report feedback. For instance, the oximetry sensor 312 may be configured to be connected to the medical device 106 described above.

The oximetry sensor 312 includes a housing 314 that at least partially encloses circuitry within the oximetry sensor 312. In various cases, the housing 314 is configured to be removably coupled with a finger of a subject. The oximetry sensor 312, for instance, includes at least one light source configured to emit light through the finger of the subject. The oximetry sensor 312 may further include at least one light sensor configured to detect light that is emitted through and/or scattered by the finger of the subject. The amount of light detected by the light sensor(s) is indicative of a blood oxygenation of the subject. Thus, the oximetry sensor 312 may detect the blood oxygenation of the subject when the oximetry sensor 312 is appropriately positioned on the subject's finger. An electrical signal (e.g., a digital signal) indicative of the blood oxygenation can be transmitted from the oximetry sensor 312 to a medical device via a cable 316.

In various implementations of the present disclosure, a light source 318 is disposed in and/or on the housing 314 of the oximetry sensor 312. The light source 318 is configured to output a light signal indicating feedback. For instance, a frequency, durations, pulse patterns, periods, colors, tones, intensities, or any combination thereof, of the light signal is indicative of the feedback.

In some cases, the circuitry within the oximetry sensor 312 (e.g., the light source(s) and light sensor(s) within the oximetry sensor 312) and the light source 318 are connected to a power source of the oximetry sensor 312, such as when the oximetry sensor 312 is disconnected from the medical device. In some examples, the circuitry and the light source 318 are electrically connected to a power source in the medical device when the oximetry sensor 312 is connected with the medical device. Thus, the circuitry in the oximetry sensor 312 and/or the light source 318 may be powered regardless of whether the oximetry sensor 312 is connected to the medical device.

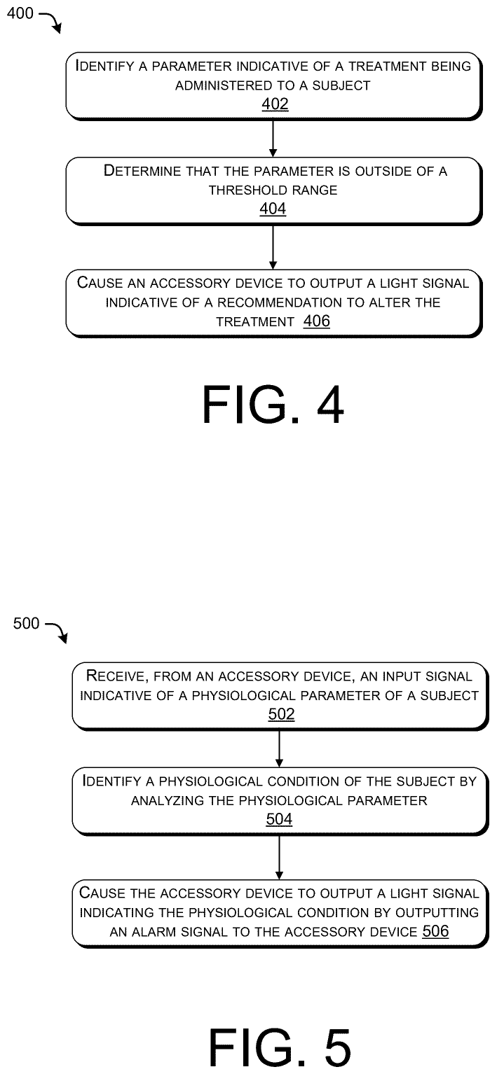

FIG. 4 illustrates an example process 400 for communicating feedback regarding a treatment via an accessory device. The process 400, in various cases, is performed by an entity, which may include at least one of a medical device (e.g., the medical device 106 and/or the medical device 202), at least one processor, a computing device, or any combination thereof.

At 402, the entity identifies a parameter indicative of a treatment being administered to a subject. In some cases, the entity identifies the parameter by analyzing a physiological parameter of the subject. Optionally, the physiological parameter is derived based on an input signal received from the accessory device itself. For instance, the physiological parameter includes electrocardiogram (ECG), a transthoracic impedance, a heart rate, a pulse rate, a capnograph, a partial pressure of CO₂ in an airway of the subject, a partial pressure of O₂ in the airway of the subject, a respiration rate, a temperature, a blood pressure, a blood oxygenation, or a plethysmograph. The treatment, in various cases, includes chest compressions, assisted ventilation, pacing, administration of an electrical shock, administration of a medication, or a combination thereof. In some cases, the treatment parameter includes a dosage, a frequency, a duty cycle, a magnitude, a location, a timing, or a combination thereof, of the treatment.

At 404, the entity determines that the parameter is outside of a threshold range. For example, the entity determines that the parameter is above a first threshold and/or below a second threshold. In various cases, the first threshold is greater than the second threshold. In some examples, the entity determines that a rate-of-change of the parameter, with respect to time, is outside of the threshold range.

At 406, the entity causes the accessory device to output a light signal indicative of a recommendation to alter the treatment. In some cases, the entity outputs an electrical signal to the accessory device, which may activate the light source. In some cases, the entity outputs a light signal to a light guide that propagates the light signal. Optionally, the light guide itself outputs at least a portion of the light signal. In some cases, the entity further causes the accessory to cease outputting the light signal. For instance, the entity may determine that an additional measurement of the treatment parameter is inside (e.g., has entered) the threshold range, and in response, may cease outputting the electrical signal and/or light signal to the accessory device.

FIG. 5 illustrates an example process 500 for communicating physiological feedback via an accessory device. The process 500, in various cases, is performed by an entity, which may include at least one of a medical device (e.g., the medical device 106 and/or the medical device 202), at least one processor, a computing device, or any combination thereof.

At 502, the entity receives, from an accessory device, an input signal indicative of a physiological parameter of a subject. For example, the accessory device includes at least one sensor configured to detect the physiological parameter and to generate the input signal based on the physiological parameter. Example physiological parameters include, for instance, an ECG, a transthoracic impedance, a heart rate, a pulse rate, a capnograph, a partial pressure of CO₂ in an airway of the subject, a partial pressure of O₂ in the airway of the subject, a respiration rate, a temperature, a blood pressure, a blood oxygenation, or a plethysmograph.

At 504, the entity identifies a physiological condition of the subject by analyzing the physiological parameter. For example, the physiological condition is indicative of an arrhythmia or other heart condition experienced by the subject. Other types of physiological conditions include a fever, a seizure, respiratory distress (e.g., lack of spontaneous breathing), lack of spontaneous circulation, high blood pressure, low blood pressure, low blood oxygenation, or the like. For instance, the entity may identify the physiological condition by comparing the physiological parameter, or a rate-of-change of the physiological parameter with respect to time, to one or more thresholds. In various cases, the entity determines the physiological condition by determining that the physiological parameter is outside of a threshold range.

At 506, the entity causes the accessory device to output a light signal indicating the physiological condition by outputting an alarm signal to the accessory device. The alarm signal, for instance, includes the light signal and/or an electrical signal. The alarm signal causes the accessory device to output the light signal, such as via a light guide or a light source within the accessory device itself.

In some cases, the entity identifies a recommended treatment associated with the physiological condition. Example treatments include, for example, administration of chest compressions, an electrical shock, pacing pulses, assisted ventilation, or a medication. For instance, the light signal indicates the recommended treatment. In some examples, the entity administers the recommended treatment to the subject. According to some cases, the entity further determines that the subject no longer exhibits the physiological condition and causes the accessory device to cease outputting the light signal in response. For example, the entity may determine that an updated measurement of the physiological parameter is within the threshold range.

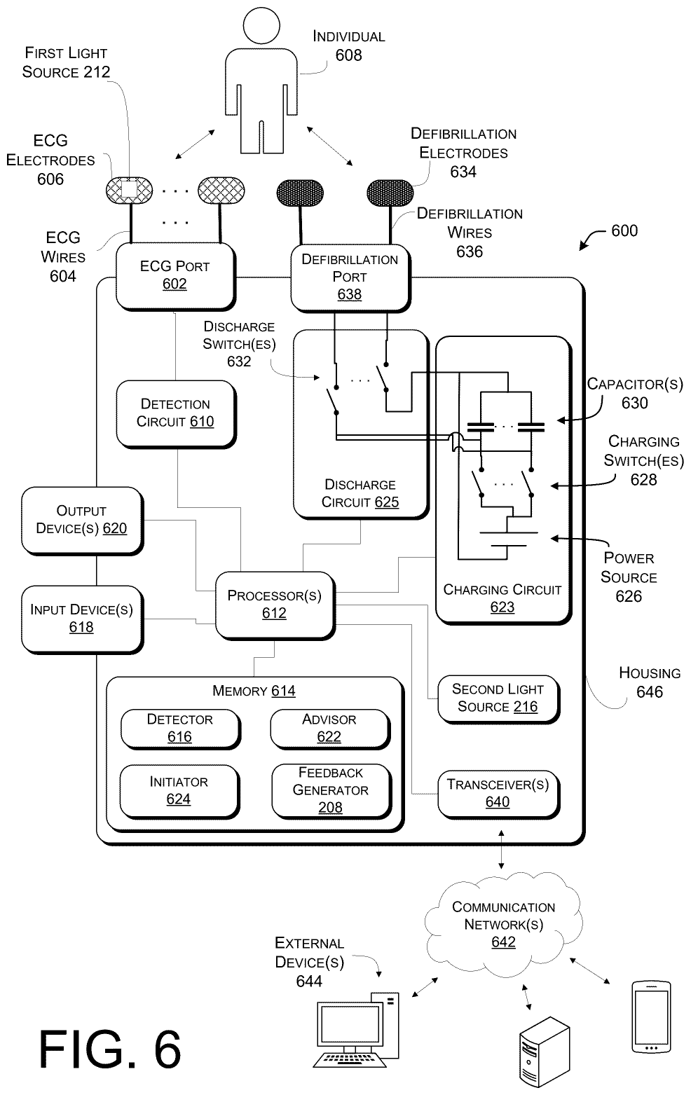

FIG. 6 illustrates an example of an external defibrillator 600 configured to perform various functions described herein. For example, the external defibrillator 600 is the medical device 106 described above with reference to FIG. 1 and/or the medical device 202 described above with reference to FIG. 2.

The external defibrillator 600 includes an electrocardiogram (ECG) port 602 connected to multiple ECG wires 604. In some cases, the ECG wires 604 are removeable from the ECG port 602. For instance, the ECG wires 604 are plugged into the ECG port 602 via connectors. The ECG wires 604 are connected to ECG electrodes 606, respectively. In various implementations, the ECG electrodes 606 are disposed on different locations on an individual 608. A detection circuit 610 is configured to detect relative voltages between the ECG electrodes 606. These voltages are indicative of the electrical activity of the heart of the individual 608.

In various implementations, the ECG electrodes 606 are in contact with the different locations on the skin of the individual 608. In some examples, a first one of the ECG electrodes 606 is placed on the skin between the heart and right arm of the individual 608, a second one of the ECG electrodes 606 is placed on the skin between the heart and left arm of the individual 608, and a third one of the ECG electrodes 606 is placed on the skin between the heart and a leg (either the left leg or the right leg) of the individual 608. In these examples, the detection circuit 610 is configured to measure the relative voltages between the first, second, and third ECG electrodes 606. Respective pairings of the ECG electrodes 606 are referred to as “leads,” and the voltages between the pairs of ECG electrodes 606 are known as “lead voltages.” In some examples, more than three ECG electrodes 606 are included, such that 5-lead or 12-lead ECG signals are detected by the detection circuit 610. In various implementations, the ECG electrodes 606 include the connection status indicator 128. For instance, the ECG electrodes 606 may include various elements and functionality of the electrode pads 108 and/or accessory device 204.

The detection circuit 610 includes at least one analog circuit, at least one digital circuit, or a combination thereof. The detection circuit 610 receives the analog electrical signals from the ECG electrodes 606, via the ECG port 602 and the ECG wires 604. In some cases, the detection circuit 610 includes one or more analog filters configured to filter noise and/or artifact from the electrical signals. The detection circuit 610 includes an analog-to-digital (ADC) in various examples. The detection circuit 610 generates a digital signal indicative of the analog electrical signals from the ECG electrodes 606. This digital signal can be referred to as an “ECG signal” or an “ECG.”

In some cases, the detection circuit 610 further detects an electrical impedance between at least one pair of the ECG electrodes 606. For example, the detection circuit 610 includes, or otherwise controls, a power source that applies a known voltage (or current) across a pair of the ECG electrodes 606 and detects a resultant current (or voltage) between the pair of the ECG electrodes 606. The impedance is generated based on the applied signal (voltage or current) and the resultant signal (current or voltage). In various cases, the impedance corresponds to respiration of the individual 608, chest compressions performed on the individual 608, and other physiological states of the individual 608. In various examples, the detection circuit 610 includes one or more analog filters configured to filter noise and/or artifact from the resultant signal. The detection circuit 610 generates a digital signal indicative of the impedance using an ADC. This digital signal can be referred to as an “impedance signal” or an “impedance.”

The detection circuit 610 provides the ECG signal and/or the impedance signal one or more processors 612 in the external defibrillator 600. In some implementations, the processor(s) 612 includes a central processing unit (CPU), a graphics processing unit (GPU), both CPU and GPU, or other processing unit or component known in the art.

The processor(s) 612 is operably connected to memory 614. In various implementations, the memory 614 is volatile (such as random access memory (RAM)), non-volatile (such as read only memory (ROM), flash memory, etc.) or some combination of the two. The memory 614 stores instructions that, when executed by the processor(s) 612, causes the processor(s) 612 to perform various operations. In various examples, the memory 614 stores methods, threads, processes, applications, objects, modules, any other sort of executable instruction, or a combination thereof. In some cases, the memory 614 stores files, databases, or a combination thereof. In some examples, the memory 614 includes, but is not limited to, RAM, ROM, electrically erasable programmable read-only memory (EEPROM), flash memory, or any other memory technology. In some examples, the memory 614 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 612 and/or the external defibrillator 600. In some cases, the memory 614 at least temporarily stores the ECG signal and/or the impedance signal.

In various examples, the memory 614 includes a detector 616, which causes the processor(s) 612 to determine, based on the ECG signal and/or the impedance signal, whether the individual 608 is exhibiting a particular heart rhythm. For instance, the processor(s) 612 determines whether the individual 608 is experiencing a shockable rhythm that is treatable by defibrillation. Examples of shockable rhythms include ventricular fibrillation (VF) and ventricular tachycardia (V-Tach). In some examples, the processor(s) 612 determines whether any of a variety of different rhythms (e.g., asystole, sinus rhythm, atrial fibrillation (AF), etc.) are present in the ECG signal.

The processor(s) 612 is operably connected to one or more input devices 618 and one or more output devices 620. Collectively, the input device(s) 618 and the output device(s) 620 function as an interface between a user and the defibrillator 600. The input device(s) 618 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 620 includes at least one of a display, a speaker, a haptic output device, a printer, or any combination thereof. In various examples, the processor(s) 612 causes a display among the input device(s) 618 to visually output a waveform of the ECG signal and/or the impedance signal. In some implementations, the input device(s) 618 includes one or more touch sensors, the output device(s) 620 includes a display screen, and the touch sensor(s) are integrated with the display screen. Thus, in some cases, the external defibrillator 600 includes a touchscreen configured to receive user input signal(s) and visually output physiological parameters, such as the ECG signal and/or the impedance signal.

In some examples, the memory 614 includes an advisor 622, which, when executed by the processor(s) 612, causes the processor(s) 612 to generate advice and/or control the output device(s) 620 to output the advice to a user (e.g., a rescuer). In some examples, the processor(s) 612 provides, or causes the output device(s) 620 to provide, an instruction to perform CPR on the individual 608. In some cases, the processor(s) 612 evaluates, based on the ECG signal, the impedance signal, or other physiological parameters, CPR being performed on the individual 608 and causes the output device(s) 620 to provide feedback about the CPR in the instruction. According to some examples, the processor(s) 612, upon identifying that a shockable rhythm is present in the ECG signal, causes the output device(s) 620 to output an instruction and/or recommendation to administer a defibrillation shock to the individual 608.

The memory 614 also includes an initiator 624 which, when executed by the processor(s) 612, causes the processor(s) 612 to control other elements of the external defibrillator 600 in order to administer a defibrillation shock to the individual 608. In some examples, the processor(s) 612 executing the initiator 624 selectively causes the administration of the defibrillation shock based on determining that the individual 608 is exhibiting the shockable rhythm and/or based on an input from a user (received, e.g., by the input device(s) 618. In some cases, the processor(s) 612 causes the defibrillation shock to be output at a particular time, which is determined by the processor(s) 612 based on the ECG signal and/or the impedance signal. In some cases, the memory 614 further includes the responder 228.

The processor(s) 612 is operably connected to a charging circuit 623 and a discharge circuit 625. Collectively, the charging circuit 623 and the discharge circuit 625 may be referred to as a “therapy circuit.” In various implementations, the charging circuit 623 includes a power source 626, one or more charging switches 628, and one or more capacitors 630. The power source 626 includes, for instance, a battery. The processor(s) 612 initiates a defibrillation shock by causing the power source 626 to charge at least one capacitor among the capacitor(s) 630. For example, the processor(s) 612 activates at least one of the charging switch(es) 628 in the charging circuit 623 to complete a first circuit connecting the power source 626 and the capacitor to be charged. Then, the processor(s) 612 causes the discharge circuit 625 to discharge energy stored in the charged capacitor across a pair of defibrillation electrodes 634, which are in contact with the individual 608. For example, the processor(s) 612 deactivates the charging switch(es) 628 completing the first circuit between the capacitor(s) 630 and the power source 626, and activates one or more discharge switches 632 completing a second circuit connecting the charged capacitor 630 and at least a portion of the individual 608 disposed between defibrillation electrodes 634.

The energy is discharged from the defibrillation electrodes 634 in the form of a defibrillation shock. For example, the defibrillation electrodes 634 are connected to the skin of the individual 608 and located at positions on different sides of the heart of the individual 608, such that the defibrillation shock is applied across the heart of the individual 608. The defibrillation shock, in various examples, depolarizes a significant number of heart cells in a short amount of time. The defibrillation shock, for example, interrupts the propagation of the shockable rhythm (e.g., VF or V-Tach) through the heart. In some examples, the defibrillation shock is 200 J or greater with a duration of about 0.015 seconds. In some cases, the defibrillation shock has a multiphasic (e.g., biphasic) waveform. The discharge switch(es) 632 are controlled by the processor(s) 612, for example. In various implementations, the defibrillation electrodes 634 are connected to defibrillation leads 636. The defibrillation wires 636 are connected to a defibrillation port 638, in implementations. According to various examples, the defibrillation wires 636 are removable from the defibrillation port 638. For example, the defibrillation wires 636 are plugged into the defibrillation port 638.

According to some cases, the memory 614 includes instructions that, when executed by the processor(s) 612, causes the processor(s) 612 to perform operations of the feedback generator 208 described above with reference to FIG. 2. Furthermore, the ECG electrodes 606 may include the first light source 212 configured to output a light signal indicating treatment and/or physiological feedback. In some cases, the defibrillator 600 itself includes the second light source 216 configured to output the light signal. In some cases, a light guide (not illustrated) is connected to, or integrated with, the ECG wires 604, such that the light signal output by the second light source 216 is viewable at the site of the ECG electrodes 606.

In various implementations, the processor(s) 612 is operably connected to one or more transceivers 640 that transmit and/or receive data over one or more communication networks 642. For example, the transceiver(s) 640 includes a network interface card (NIC), a network adapter, a local area network (LAN) adapter, or a physical, virtual, or logical address to connect to the various external devices and/or systems. In various examples, the transceiver(s) 640 includes any sort of wireless transceivers capable of engaging in wireless communication (e.g., radio frequency (RF) communication). For example, the communication network(s) 642 includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 640 includes other wireless modems, such as a modem for engaging in WI-FI®, WIGIG®, WIMAX®, BLUETOOTH®, or infrared communication over the communication network(s) 642.

The defibrillator 600 is configured to transmit and/or receive data (e.g., ECG data, impedance data, data indicative of one or more detected heart rhythms of the individual 608, data indicative of one or more defibrillation shocks administered to the individual 608, etc.) with one or more external devices 644 via the communication network(s) 642. The external devices 644 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 642. In some examples, the external device(s) 644 is located remotely from the defibrillator 600, such as at a remote clinical environment (e.g., a hospital). According to various implementations, the processor(s) 612 causes the transceiver(s) 640 to transmit data to the external device(s) 644. In some cases, the transceiver(s) 640 receives data from the external device(s) 644 and the transceiver(s) 640 provide the received data to the processor(s) 612 for further analysis.

In various implementations, the external defibrillator 600 also includes a housing 646 that at least partially encloses other elements of the external defibrillator 600. For example, the housing 646 encloses the detection circuit 610, the processor(s) 612, the memory 614, the charging circuit 623, the transceiver(s) 640, or any combination thereof. In some cases, the input device(s) 618 and output device(s) 620 extend from an interior space at least partially surrounded by the housing 646 through a wall of the housing 646. In various examples, the housing 646 acts as a barrier to moisture, electrical interference, and/or dust, thereby protecting various components in the external defibrillator 600 from damage.

In some implementations, the external defibrillator 600 is an automated external defibrillator (AED) operated by an untrained user (e.g., a bystander, layperson, etc.) and can be operated in an automatic mode. In automatic mode, the processor(s) 612 automatically identifies a rhythm in the ECG signal, makes a decision whether to administer a defibrillation shock, charges the capacitor(s) 630, discharges the capacitor(s) 630, or any combination thereof. In some cases, the processor(s) 612 controls the output device(s) 620 to output (e.g., display) a simplified user interface to the untrained user. For example, the processor(s) 612 refrains from causing the output device(s) 620 to display a waveform of the ECG signal and/or the impedance signal to the untrained user, in order to simplify operation of the external defibrillator 600.

In some examples, the external defibrillator 600 is a monitor-defibrillator utilized by a trained user (e.g., a clinician, an emergency responder, etc.) and can be operated in a manual mode or the automatic mode. When the external defibrillator 600 operates in manual mode, the processor(s) 612 cause the output device(s) 620 to display a variety of information that may be relevant to the trained user, such as waveforms indicating the ECG data and/or impedance data, notifications about detected heart rhythms, and the like.

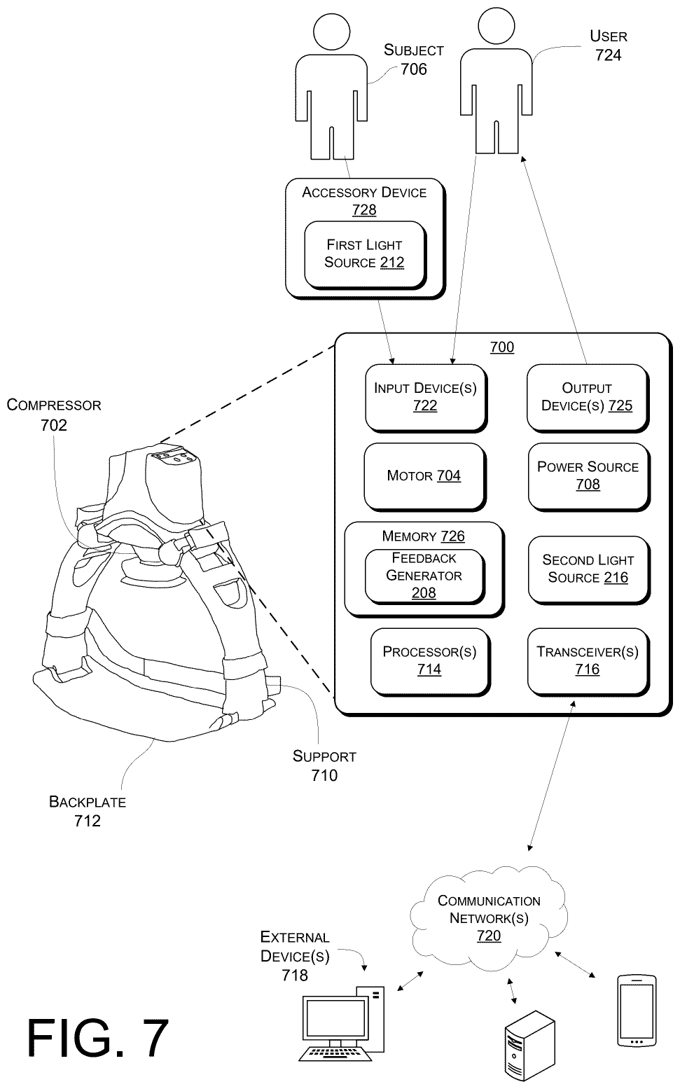

FIG. 7 illustrates a chest compression device 700 configured to perform various functions described herein. For example, the chest compression device 700 is the medical device 202 described in FIG. 2.

In various implementations, the chest compression device 700 includes a compressor 702 that is operatively coupled to a motor 704. The compressor 702 physically administers a force to the chest of a subject 706 that compresses the chest of the subject 706. In some examples, the compressor 702 includes at least one piston that periodically moves between two positions (e.g., a compressed position and a release position) at a compression frequency. For example, when the piston is positioned on the chest of the subject 706, the piston compresses the chest when the piston is moved into the compressed position. A suction cup may be positioned on a tip of the piston, such that the suction cup contacts the chest of the subject 706 during operation. In various cases, the compressor 702 includes a band that periodically tightens to a first tension and loosens to a second tension at a compression frequency. For instance, when the band is disposed around the chest of the subject 706, the band compresses the chest when the band tightens.

The motor 704 is configured to convert electrical energy stored in a power source 708 into mechanical energy that moves and/or tightens the compressor 702, thereby causing the compressor 702 to administer the force to the chest of the subject 706. In various implementations, the power source 708 is portable. For instance, the power source 708 includes at least one rechargeable (e.g., lithium-ion) battery. In some cases, the power source 708 supplies electrical energy to one or more elements of the chest compression device 700 described herein.

In various cases, the chest compression device 700 includes a support 710 that is physically coupled to the compressor 702, such that the compressor 702 maintains a position relative to the subject 706 during operation. In some implementations, the support 710 is physically coupled to a backplate 712, cot, or other external structure with a fixed position relative to the subject 706. According to some cases, the support 710 is physically coupled to a portion of the subject 706, such as wrists of the subject 706.

The operation of the chest compression device 700 may be controlled by at least one processor 714. In various implementations, the motor 704 is communicatively coupled to the processor(s) 714. Specifically, the processor(s) 714 is configured to output a control signal to the motor 704 that causes the motor 704 to actuate the compressor 702. For instance, the motor 704 causes the compressor 702 to administer the compressions to the subject 706 based on the control signal. In some cases, the control signal indicates one or more treatment parameters of the compressions. Examples of treatment parameters include a frequency, timing, depth, force, position, velocity, and acceleration of the compressor 702 administering the compressions. According to various cases, the control signal causes the motor 704 to cease compressions.

In various implementations, the chest compression device 700 includes at least one transceiver 716 configured to communicate with at least one external device 718 over one or more communication networks 720. Any communication network described herein can be included in the communication network(s) 720 illustrated in FIG. 7. The external device(s) 718, for example, includes at least one of a monitor-defibrillator, an AED, an ECMO device, a ventilation device, a patient monitor, a mobile phone, a server, or a computing device. In some implementations, the transceiver(s) 716 is configured to communicate with the external device(s) 718 by transmitting and/or receiving signals wirelessly. For example, the transceiver(s) 716 includes a NIC, a network adapter, a LAN adapter, or a physical, virtual, or logical address to connect to the various external devices and/or systems. In various examples, the transceiver(s) 716 includes any sort of wireless transceivers capable of engaging in wireless communication (e.g., RF communication). For example, the communication network(s) 720 includes one or more wireless networks that include a 3GPP network, such as an LTE RAN (e.g., over one or more LTE bands), an NR RAN (e.g., over one or more NR bands), or a combination thereof. In some cases, the transceiver(s) 716 includes other wireless modems, such as a modem for engaging in WI-FI®, WIGIG®, WIMAX®, BLUETOOTH®, or infrared communication over the communication network(s) 720. The signals, in various cases, encode data in the form of data packets, datagrams, or the like. In some cases, the signals are transmitted as compressions are being administered by the chest compression device 700 (e.g., for real-time feedback by the external device(s) 718), after compressions are administered by the chest compression device 700 (e.g., for post-event review at the external device 718), or a combination thereof.

In various cases, the processor(s) 714 generates the control signal based on data encoded in the signals received from the external device(s) 718. For instance, the signals include an instruction to initiate the compressions, and the processor(s) 714 instructs the motor 704 to begin actuating the compressor 702 in accordance with the signals.

In some cases, the chest compression device 700 includes at least one input device 722. In various examples, the input device(s) 722 is configured to receive an input signal from a user 724, who may be a rescuer treating the subject 706. Examples of the input device(s) 722 include, for instance, at a keypad, a cursor control, a touch-sensitive display, a voice input device (e.g., a microphone), a haptic feedback device (e.g., a gyroscope), or any combination thereof. In various implementations, the processor(s) 714 generate the control signal based on the input signal. For instance, the processor(s) 714 generate the control signal to adjust a frequency of the compressions based on the chest compression device 700 detecting a selection by the user 724 of a user interface element displayed on a touchscreen or detecting the user 724 pressing a button integrated with an external housing of the chest compression device 700.

According to some examples, the input device(s) 722 include one or more sensors. The sensor(s), for example, is configured to detect a physiological parameter of the subject 706. In some implementations, the sensor(s) is configured to detect a state parameter of the chest compression device 700, such as a position of the compressor 702 with respect to the subject 706 or the backplate 712, a force administered by the compressor 702 on the subject 706, a force administered onto the backplate 712 by the body of the subject 706 during a compression, or the like. According to some implementations, the signals transmitted by the transceiver(s) 716 indicate the physiological parameter(s) and/or the state parameter(s).

The chest compression device 700 further includes at least one output device 725, in various implementations. Examples of the output device(s) 725 include, for instance, least one of a display (e.g., a projector, an LED screen, etc.), a speaker, a haptic output device, a printer, or any combination thereof. In some implementations, the output device(s) 725 include a screen configured to display various parameters detected by and/or reported to the chest compression device 700, a charge level of the power source 708, a timer indicating a time since compressions were initiated or paused, and other relevant information.

The chest compression device 700 further includes memory 726. In various implementations, the memory 726 is volatile (such as random access memory (RAM)), non-volatile (such as read only memory (ROM), flash memory, etc.) or some combination of the two. The memory 726 stores instructions that, when executed by the processor(s) 714, causes the processor(s) 714 to perform various operations. In various examples, the memory 726 stores methods, threads, processes, applications, objects, modules, any other sort of executable instruction, or a combination thereof. In some cases, the memory 726 stores files, databases, or a combination thereof. In some examples, the memory 726 includes, but is not limited to, RAM, ROM, EEPROM, flash memory, or any other memory technology. In some examples, the memory 726 includes one or more of CD-ROMs, DVDs, CAM, or other optical storage, magnetic cassettes, magnetic tape, magnetic disk storage or other magnetic storage devices, or any other medium which can be used to store the desired information. In various cases, the memory 726 stores instructions, programs, threads, objects, data, or any combination thereof, that cause the processor(s) 714 to perform various functions. In various cases, the memory 726 stores one or more parameters that are detected by the chest compression device 700 and/or reported to the chest compression device 700. In some cases, the memory 726 includes the feedback generator 208.

In implementations of the present disclosure, an accessory device 728 is removably connected to the chest compression device 700. In some cases, the accessory device 728 is configured to output a light signal indicating treatment and/or physiological feedback of the subject 706 to the user. For example, the accessory device 728 includes the first light source 212. In some examples, the chest compression device 700 itself includes the second light source 216, which may transmit the light signal to the accessory device 728 via a light guide (not illustrated).

### EXAMPLE CLAUSES

The following clauses provide examples of various implementations of the present disclosure.
1. A system, including: a medical device including: a port; a measurement circuit configured to: detect transthoracic impedance of a subject by analyzing an input signal received by the port; a processor configured to: identify a rate of chest compressions administered to the subject by analyzing the transthoracic impedance; determine that the rate of the chest compressions is outside of a threshold range; in response to determining that the rate of the chest compressions is outside of the threshold range, generate an output signal; and provide the output signal to the port; and an electrode accessory including: a connector configured to be removably coupled with the port, the connector including a light source configured to output a visual signal in response to receiving the output signal, wherein a color, frequency, or pulse pattern of the visual signal is indicative of a recommendation to alter the rate of the chest compressions; an electrode pad including: an electrically insulative substrate; and an electrode disposed on a first surface of the electrically insulative substrate and configured to detect the input signal from a chest of the subject; a cable configured to electrically connect the connector to the electrode; and a light guide optically connected to the light source and configured to propagate the visual signal, the light guide extending along the cable and onto a second surface of the electrically insulative substrate.
2. The system of clause 1, the output signal being a first output signal, the visual signal being a first visual signal, wherein the processor is further configured to: determine that the rate of the chest compressions has entered the threshold range; in response to determining that the rate of the chest compressions has entered the threshold range, generate a second output signal; and provide the second output signal to the port, and wherein the light source is further configured to output a second visual signal in response to receiving the second output signal, wherein a color, frequency, or pulse pattern of the second visual signal is indicative of a recommendation to maintain the rate of the chest compressions, the color, frequency, or pulse pattern of the first visual signal being different than the color, frequency, or pulse pattern of the second visual signal.
3. The system of clause 1 or 2, wherein a frequency of the pulse pattern of the visual signal is within the threshold range.
4. A medical device including: a port configured to be removably coupled with an accessory device; a processor configured to: identify a treatment parameter indicative of a treatment being administered to a subject; determine that the treatment parameter is outside of a threshold range; and in response to determining that the treatment parameter is outside of the threshold range, cause the accessory device to output a visual signal indicative of a recommendation to alter the treatment by providing an output signal to the port.
5. The medical device of clause 4, wherein the port is further configured to receive, from the accessory device, an input signal, and wherein the processor is configured to determine a physiological parameter of the subject by analyzing the input signal.
6. The medical device of clause 5, wherein the physiological parameter of the subject includes an electrocardiogram (ECG), a transthoracic impedance, a heart rate, a pulse rate, a capnograph, a partial pressure of CO₂ in an airway of the subject, a partial pressure of O₂ in the airway of the subject, a respiration rate, a temperature, a blood pressure, a blood oxygenation, or a plethysmograph.
7. The medical device of clause 5 or 6, wherein the processor is configured to identify the treatment parameter indicative of the treatment being administered to the subject by analyzing the physiological parameter of the subject.
8. The medical device of any of clauses 4 to 7, further including: a transceiver configured to receive a communication signal indicating the treatment parameter; or a sensor configured to detect a signal indicating the treatment parameter.
9. The medical device of any of clauses 4 to 8, wherein the treatment includes chest compressions, assisted ventilation, pacing, administration of an electrical shock, or administration of a medication.
10. The medical device of any of clauses 4 to 9, wherein the treatment parameter includes a dosage, a frequency, a duty cycle, a magnitude, a location, or a timing of the treatment.
11. The medical device of any of clauses 4 to 10, wherein the output signal includes an electrical signal that activates a light source in the accessory device.
12. The medical device of any of clauses 4 to 11, wherein the port includes a light source, the output signal causing the light source to output the visual signal, and wherein the port is configured to be coupled with a light guide in the accessory device, the light guide being configured to propagate the visual signal and to output a portion of the visual signal.
13. A method, including: identifying a treatment parameter indicative of a treatment being administered to a subject; determining that the treatment parameter is outside of a threshold range; and in response to determining that the treatment parameter is outside of the threshold range, causing an accessory device to output a visual signal indicative of a recommendation to alter the treatment.
14. The method of clause 13, further including: receiving an input signal from the accessory device; determining a physiological parameter of the subject by analyzing the input signal; and determining the treatment parameter by analyzing the physiological parameter.
15. The method of clause 14, wherein the physiological parameter of the subject includes an electrocardiogram (ECG), a transthoracic impedance, a heart rate, a pulse rate, a capnograph, a partial pressure of CO₂ in an airway of the subject, a partial pressure of O₂ in the airway of the subject, a respiration rate, a temperature, a blood pressure, a blood oxygenation, or a plethysmograph.
16. The method of any of clauses 13 to 15, wherein the treatment includes chest compressions, assisted ventilation, pacing, administration of an electrical shock, or administration of a medication.
17. The method of any of clauses 13 to 16, wherein the treatment parameter includes a dosage, a frequency, a duty cycle, a magnitude, a location, or a timing of the treatment.
18. The method of any of clauses 13 to 17, wherein causing the accessory device to output a visual signal indicative of a recommendation to alter the treatment includes outputting an electrical signal that activates a light source in the accessory device.
19. The method of any of clauses 13 to 18, wherein causing the accessory device to output the visual signal indicative of the recommendation to alter the treatment includes outputting the visual signal to a light guide of the accessory device, the light guide being configured to propagate the visual signal and to output a portion of the visual signal.
20. The method of any of clauses 13 to 19, further including: determining that the treatment parameter is inside of the threshold range; and in response to determining that the treatment parameter is inside of the threshold range, causing an accessory device to cease outputting the visual signal.
21. A system, including: a medical device including: a port; a measurement circuit configured to detect an electrocardiogram (ECG) of a subject by analyzing an input signal received by the port; a processor configured to: determine that the ECG is indicative of ventricular fibrillation (VF); in response to determining that the ECG is indicative of VF, generate an alarm signal; and provide the alarm signal to the port; and an electrode accessory including: a connector configured to be removably coupled with the port, the connector including a light source configured to output a visual signal in response to receiving the alarm signal, wherein a color, frequency, or pulse pattern of the visual signal is indicative of a recommendation to administer an electrical shock to the subject; an electrode pad including: an electrically insulative substrate; and an electrode disposed on a first surface of the electrically insulative substrate and configured to detect the input signal from a chest of the subject; a cable configured to electrically connect the connector to the electrode; and a light guide optically connected to the light source and configured to propagate the visual signal, the light guide extending along the cable and onto a second surface of the electrically insulative substrate.
22. The system of clause 21, wherein the medical device is an external defibrillator further including: a therapy circuit configured to output the electrical shock to the electrode via the port; and an input device configured to detect a user input signal, and wherein the processor is further configured to cause the therapy circuit to output the electrical shock to the electrode via the port in response to the input device detecting the user input signal.
23. The system of clause 21 or 22, further including: a display configured to indicate the VF.
24. An accessory device, including: a sensor configured to detect a physiological parameter of a subject; and a light source configured to output a visual signal in response to the accessory device receiving an alarm signal from a medical device, the visual signal indicating a physiological condition of the subject.
25. The accessory device of clause 24, wherein the sensor includes an electrode, multiple electrodes, a pulse sensor, a capnography sensor, a gas sensor, a temperature sensor, a blood pressure sensor, a motion sensor, a light sensor, or an oxygenation sensor.
26. The accessory device of clause 24 or 25, wherein the physiological parameter of the subject includes an electrocardiogram (ECG), a transthoracic impedance, a heart rate, a pulse rate, a capnograph, a partial pressure of CO₂ in an airway of the subject, a partial pressure of O₂ in the airway of the subject, a respiration rate, a temperature, a blood pressure, a blood oxygenation, or a plethysmograph.
27. The accessory device of any of clauses 24 to 26, further including: a light guide configured to propagate the visual signal and output a portion of the visual signal.
28. The accessory device of clause 27, further including: a connector configured to be removably coupled with a port of the medical device and to receive the alarm signal from the port of the medical device; and a cable electrically coupled between the connector and the sensor and configured to transmit, to the port of the medical device, an input signal indicative of the physiological parameter, the light guide being physically coupled with the cable.
29. The accessory device of any of clauses 24 to 28, wherein the physiological condition includes the physiological parameter being outside of a threshold range.
30. The accessory device of any of clauses 24 to 29, wherein the visual signal further indicates a recommended treatment to administer to the subject, the recommended treatment including an electrical shock, pacing pulses, chest compressions, or administration of a medication.
31. The accessory device of any of clauses 24 to 30, wherein a color, frequency, pulse pattern, or intensity of the visual signal indicates the physiological condition of the subject.
32. The accessory device of any of clauses 24 to 31, further including: a transceiver configured to: transmit, to the medical device, a first communication signal indicative of the physiological parameter; and receive, from the medical device, a second communication signal indicative of the alarm signal.
33. A method, including: receiving, from an accessory device, an input signal indicative of a physiological parameter of a subject; identifying a physiological condition of the subject by analyzing the physiological parameter; and causing the accessory device to output a visual signal indicating the physiological condition by outputting an alarm signal to the accessory device.
34. The method of clause 33, wherein the physiological parameter of the subject includes an electrocardiogram (ECG), a transthoracic impedance, a heart rate, a pulse rate, a capnograph, a partial pressure of CO₂ in an airway of the subject, a partial pressure of O₂ in the airway of the subject, a respiration rate, a temperature, a blood pressure, a blood oxygenation, or a plethysmograph.
35. The method of clause 33 or 34, wherein identifying the physiological condition of the subject includes determining that the physiological parameter is indicative of an arrhythmia.
36. The method of any of clauses 33 to 35, wherein identifying the physiological condition of the subject includes determining that the physiological parameter is outside of a threshold range.
37. The method of any of clauses 33 to 36, wherein the alarm signal includes the visual signal or an electrical signal.
38. The method of any of clauses 33 to 37, further including: identifying a recommended treatment associated with the physiological condition, wherein the visual signal further indicates the recommended treatment.
39. The method of clause 38, further including: administering the recommended treatment to the subject.
40. The method of any of clauses 33 to 39, further including: determining that the subject no longer exhibits the physiological condition; and in response to determining that the subject no longer exhibits the physiological condition, causing the accessory device to cease outputting the visual signal indicating the physiological condition.
41. A method, including: determining that a physiological parameter detected by an accessory device is unreliable; and in response to determining that the physiological parameter detected by the accessory device is unreliable, causing output of a visual signal associated with the accessory device.
42. The method of clause 41, wherein determining that the physiological parameter detected by the accessory device is unreliable includes detecting that data indicative of the physiological parameter includes an artifact.
43. The method of clause 41 or 42, wherein determining that the physiological parameter detected by the accessory device is unreliable includes determining that a signal-to-noise ratio of data indicative of the physiological parameter is below a threshold.
44. The method of any of clauses 41 to 43, wherein causing the output of the visual signal associated with the accessory device includes illuminating a light source optically coupled with a light guide that is physically connected to the accessory device.
45. The method of any of clauses 41 to 44, causing the output of the visual signal associated with the accessory device includes causing a light source of the accessory device to output the visual signal.
46. The method of any of clauses 41 to 45, wherein the accessory device includes electrode, multiple electrodes, a pulse sensor, a capnography sensor, a gas sensor, a temperature sensor, a blood pressure sensor, a motion sensor, a light sensor, or an oxygenation sensor.
47. The method of any of clauses 41 to 46, wherein the physiological parameter includes an electrocardiogram (ECG), a transthoracic impedance, a heart rate, a pulse rate, a capnograph, a partial pressure of CO₂ in an airway of a subject, a partial pressure of O₂ in the airway of the subject, a respiration rate, a temperature, a blood pressure, a blood oxygenation, or a plethysmograph.
48. The method of any of clauses 41 to 47, wherein determining that the physiological parameter detected by the accessory device is unreliable includes determining a magnitude of unreliability of the accessory device, and wherein a characteristic of the visual signal is indicative of the magnitude of unreliability.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A system, comprising:
a medical device comprising:
a port;
a measurement circuit configured to: detect transthoracic impedance of a subject by analyzing an input signal received by the port;
a processor configured to:
identify a rate of chest compressions administered to the subject by analyzing the transthoracic impedance;
determine that the rate of the chest compressions is outside of a threshold range;
in response to determining that the rate of the chest compressions is outside of the threshold range, generate an output signal; and
provide the output signal to the port; and
an electrode accessory comprising:
a connector configured to be removably coupled with the port, the connector comprising a light source configured to output a visual signal in response to receiving the output signal, wherein a color, frequency, or pulse pattern of the visual signal is indicative of a recommendation to alter the rate of the chest compressions;
an electrode pad comprising:
an electrically insulative substrate; and
an electrode disposed on a first surface of the electrically insulative substrate and configured to detect the input signal from a chest of the subject;
a cable configured to electrically connect the connector to the electrode; and
a light guide optically connected to the light source and configured to propagate the visual signal, the light guide extending along the cable and onto a second surface of the electrically insulative substrate.

2. The system of claim 1, the output signal being a first output signal, the visual signal being a first visual signal, wherein the processor is further configured to:
determine that the rate of the chest compressions has entered the threshold range;
in response to determining that the rate of the chest compressions has entered the threshold range, generate a second output signal; and
provide the second output signal to the port, and
wherein the light source is further configured to output a second visual signal in response to receiving the second output signal, wherein a color, frequency, or pulse pattern of the second visual signal is indicative of a recommendation to maintain the rate of the chest compressions, the color, frequency, or pulse pattern of the first visual signal being different than the color, frequency, or pulse pattern of the second visual signal.

3. The system of claim 1, wherein a frequency of the pulse pattern of the visual signal is within the threshold range.

4. A medical device comprising:
a port configured to be removably coupled with an accessory device;
a processor configured to:
identify a treatment parameter indicative of a treatment being administered to a subject;
determine that the treatment parameter is outside of a threshold range; and
in response to determining that the treatment parameter is outside of the threshold range, cause the accessory device to output a visual signal indicative of a recommendation to alter the treatment by providing an output signal to the port.

5. The medical device of claim 4, wherein the port is further configured to receive, from the accessory device, an input signal, and
wherein the processor is configured to determine a physiological parameter of the subject by analyzing the input signal.

6. The medical device of claim 5, wherein the physiological parameter of the subject comprises an electrocardiogram (ECG), a transthoracic impedance, a heart rate, a pulse rate, a capnograph, a partial pressure of CO₂ in an airway of the subject, a partial pressure of O₂ in the airway of the subject, a respiration rate, a temperature, a blood pressure, a blood oxygenation, or a plethysmograph.

7. The medical device of claim 5, wherein the processor is configured to identify the treatment parameter indicative of the treatment being administered to the subject by analyzing the physiological parameter of the subject.

8. The medical device of claim 4, further comprising:
a transceiver configured to receive a communication signal indicating the treatment parameter; or
a sensor configured to detect a signal indicating the treatment parameter.

9. The medical device of claim 4, wherein the treatment comprises chest compressions, assisted ventilation, pacing, administration of an electrical shock, or administration of a medication.

10. The medical device of claim 4, wherein the treatment parameter comprises a dosage, a frequency, a duty cycle, a magnitude, a location, or a timing of the treatment.

11. The medical device of claim 4, wherein the output signal comprises an electrical signal that activates a light source in the accessory device.

12. The medical device of claim 4, wherein the port comprises a light source, the output signal causing the light source to output a light signal, the visual signal comprising the light signal, and
wherein the port is configured to be coupled with a light guide in the accessory device, the light guide being configured to propagate the light signal and output a portion of the light signal.

13. A method, comprising:
identifying a treatment parameter indicative of a treatment being administered to a subject;
determining that the treatment parameter is outside of a threshold range; and
in response to determining that the treatment parameter is outside of the threshold range, causing an accessory device to output a visual signal indicative of a recommendation to alter the treatment.

14. The method of claim 13, further comprising:
receiving an input signal from the accessory device;
determining a physiological parameter of the subject by analyzing the input signal; and
determining the treatment parameter by analyzing the physiological parameter.

15. The method of claim 14, wherein the physiological parameter of the subject comprises an electrocardiogram (ECG), a transthoracic impedance, a heart rate, a pulse rate, a capnograph, a partial pressure of CO₂ in an airway of the subject, a partial pressure of O₂ in the airway of the subject, a respiration rate, a temperature, a blood pressure, a blood oxygenation, or a plethysmograph.

16. The method of claim 13, wherein the treatment comprises chest compressions, assisted ventilation, pacing, administration of an electrical shock, or administration of a medication.

17. The method of claim 13, wherein the treatment parameter comprises a dosage, a frequency, a duty cycle, a magnitude, a location, or a timing of the treatment.

18. The method of claim 13, wherein causing the accessory device to output a visual signal indicative of a recommendation to alter the treatment comprises outputting an electrical signal that activates a light source in the accessory device.

19. The method of claim 13, wherein causing the accessory device to output the visual signal indicative of the recommendation to alter the treatment comprises outputting the visual signal to a light guide of the accessory device, the light guide being configured to propagate the visual signal and to output a portion of the visual signal.

20. The method of claim 13, further comprising:
determining that the treatment parameter is inside of the threshold range; and
in response to determining that the treatment parameter is inside of the threshold range, causing an accessory device to cease outputting the visual signal.
